(19) United States
(12) Patent Application Publication
(10) Pub. No.: US 2009/0092567 A1
(43) Pub. Date: Apr. 9, 2009

(54) OIL-CONTAINING COMPOSITIONS EXHIBITING REDUCED FEATHERING

(75) Inventors: Victoria Chou, Washington, DC (US); Michell Chen, Edison, NJ (US); Padraig McDermott, Meudon (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL S.A., Paris (FR)

(21) Appl. No.: 12/238,960

(22) Filed: Sep. 26, 2008

Related U.S. Application Data
(60) Provisional application No. 60/977,810, filed on Oct. 5, 2007.

(51) Int. Cl. A61K 8/72

(52) U.S. Cl. 424/64; 424/70.6

(57) ABSTRACT

The invention relates to compositions containing at least one oil, at least one silicone elastomer, at least one wax, at least one filler, and at least one coloring agent as well as to methods of making and using such compositions.

### CROSS-REFERENCE TO PRIOR APPLICATIONS

This application claims priority to U.S. provisional application 60/977,810 filed Oct. 5, 2007, the entirety of which is hereby incorporated by reference.

### FIELD OF THE INVENTION

The present invention generally relates to compositions for application to keratin materials (e.g., skin or lips) such as, for example, lipsticks, foundations, topcoats and basecoats comprising at least one oil, at least one silicone elastomer, at least one wax, at least one filler, and at least one coloring agent. Such compositions, when applied to keratin materials, possess improved properties and characteristics such as, for example, reduced feathering (oil migration), decreased drag upon application, improved sensation upon application (for example, decreased sensation of cakiness and improved feelings of smoothness and silkiness), decreased opaqueness, and/or improved satin-to-matte appearance.

### BACKGROUND OF THE INVENTION

Many lipsticks, foundations and other cosmetic compositions have been developed for improved wear and transfer-resistance properties. Typically, such compositions, particularly lipsticks, are formulated to deliver a shiny appearance to the keratin material. That is, satin or matte finishes for these types of products are generally disfavored. Part of the problem with such satin-to-matte compositions is that the means by which the satin-to-matte finish is achieved results in the compositions having detrimental properties or characteristics. For example, one way to achieve a satin-to-matte finish is to include a substantial amount of clay materials, particularly kaolin, talc and/or bentonite, in the composition. Although these materials provide the composition with a matte finish, these materials also unacceptably increase drag of the composition upon application (that is, the composition is more difficult to apply to keratin material because it “drags” across the keratin material rather than gliding across the material) and unacceptably increase the sensation of cakiness upon application (that is, the composition feels thick and dry upon the keratin material instead of smooth and silky). Also, the compositions are unacceptable opaque due to the high content of clay material.

Furthermore, cosmetic compositions containing a high amount of oil typically have problems associated with oil migration, sometimes referred to as “bleeding” or “feathering.” That is, it has been difficult in the past to prepare cosmetic compositions containing high amounts of oil which do not bleed or feather, particularly solid compositions in the form of a stick.

Thus, there remains a need for improved cosmetic compositions, particularly lipsticks and foundations, for application to keratin materials, which possess significantly improved cosmetic properties, particularly with respect to drag, cakiness, opaqueness, feathering, bleeding and satin-to-matte appearance.

Accordingly, one aspect of the present invention is a makeup, care and/or treatment compositions, particularly solid lipsticks and foundations, capable of imparting satin-to-matte appearance to keratin materials, which have significantly improved cosmetic properties.

### SUMMARY OF THE INVENTION

The present invention relates to compositions for application to keratin materials (e.g., skin or lips) such as lipsticks, foundations, topcoats and basecoats comprising at least one oil, at least one silicone elastomer, at least one wax, at least one filler, and at least one coloring agent. Preferably, such compositions are solid and substantially anhydrous, and further comprise a film forming agent or structuring agent. Also preferably, the compositions contain more non-silicone oil than silicone oil.

The present invention also relates to compositions for application to keratin materials (e.g., skin or lips) such as lipsticks, foundations, topcoats and basecoats comprising at least one oil, at least one silicone elastomer, at least one wax, at least one filler, and at least one coloring agent, wherein the total amount of oil present is at least about 30% by weight with respect to the total weight of the composition and the combined total amount of silicone elastomer(s), wax(es), filler(s) and coloring agent(s) present is at least about 30% by weight with respect to the total weight of the composition. Preferably, such compositions are solid and substantially anhydrous, and further comprise a film forming agent or structuring agent. Also preferably, the compositions contain more non-silicone oil than silicone oil.

The present invention further relates to methods of producing a composition capable of imparting a satin-to-matte appearance to keratin materials comprising combining at least one oil, at least one silicone elastomer, at least one wax, at least one filler, and at least one coloring agent to form a composition. Preferably, such compositions are solid and substantially anhydrous, and further comprise a film forming agent or structuring agent. Also preferably, the compositions contain more non-silicone oil than silicone oil.

The present invention also relates to methods of delivering a satin-to-matte appearance to a keratin material comprising applying a composition comprising at least one oil, at least one silicone elastomer, at least one wax, at least one filler, and at least one coloring agent to the keratin material in an amount sufficient to deliver a satin-to-matte appearance to a keratin material. Preferably, such compositions are solid and substantially anhydrous, and further comprise a film forming agent or structuring agent. Also preferably, the compositions contain more non-silicone oil than silicone oil.

The present invention also relates to methods of minimizing drag or cakey feeling associated with application of compositions which impart a satin-to-matte appearance to keratin materials comprising combining at least one oil, at least one silicone elastomer, at least one wax, at least one filler, and at least one coloring agent to form a composition capable of imparting a satin-to-matte appearance to keratin materials. Preferably, such compositions are solid and substantially anhydrous, and further comprise a film forming agent or structuring agent. Also preferably, the compositions contain more non-silicone oil than silicone oil.

The present invention also relates to methods of minimizing bleeding, feathering or oil migration associated with cosmetic compositions, preferably solid cosmetic compositions, comprising at least about 30% oil comprising combining at least 30% oil, at least one silicone elastomer, at least one wax, at least one filler, and at least one coloring agent to form a composition. Preferably, such compositions are solid and substantially anhydrous, and further comprise a film forming agent or structuring agent. Also preferably, the compositions contain more non-silicone oil than silicone oil.

The present invention also relates to methods of treating or caring for keratin materials by applying compositions of the present invention to the keratin materials in an amount sufficient to treat and/or care for the keratin materials.

The present invention further relates to methods of enhancing the appearance of keratin materials by applying compositions of the present invention to the keratin materials in an amount sufficient to enhance the appearance of the keratin materials.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

“Film former” or “film forming agent” as used herein means a polymer or resin that leaves a film on the substrate to which it is applied, for example, after a solvent accompanying the film former has evaporated, absorbed into and/or dissipated on the substrate.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion, and is typically reported in units of force time (for example, gram seconds (“g s”)). For example, the TA-XT2 from Stable Micro Systems, Ltd. can be used to determine adhesion following the procedures set forth in the TA-XT2 Application Study (ref: MATI/PO.25), revised January 2000, the entire contents of which are hereby incorporated by reference. According to this method, desirable values for work of adhesion for substantially non-tacky substances include less than about 0.5 g s, less than about 0.4 g s, less than about 0.3 g s and less than about 0.2 g s. As known in the art, other similar methods can be used on other similar analytical devices to determine adhesion.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes, skin, etc. In a preferred embodiment of the present invention, little or no composition is transferred from the wearer.

The cosmetic compositions and methods of the present invention can comprise, consist of, or consist essentially of the essential elements and limitations of the invention described herein, as well as any additional or optional ingredients, components, or limitations described herein or otherwise useful in personal care compositions intended for topical application to hair.

The composition of the present invention may be in any form, either liquid or non-liquid (semi-solid, soft solid, solid, etc.). For example, it may be a paste, a solid, a gel, or a cream. It may be an emulsion, such as an oil-in-water or water-in-oil emulsion, a multiple emulsion, such as an oil-in-water-in-oil emulsion or a water-in-oil-in-water emulsion, or a solid, rigid or supple gel, including anhydrous gels. The composition can also be in a form chosen from a translucent anhydrous gel and a transparent anhydrous gel. The composition of the invention may, for example, comprise an external or continuous fatty phase. The composition may be anhydrous. The composition can also be a molded composition or cast as a stick or a dish. The composition in one embodiment is a solid such as a molded stick or a poured stick.

Depending on the intended application, such as a stick, hardness of the composition may also be considered. The hardness of a composition may, for example, be expressed in gramforce (gf). The composition of the present invention may, for example, have a hardness ranging from 20 gf to 2000 gf, such as from 20 gf to 500 gf, such as and further such as from 20 gf to 300 gf, such as from 35 gf to 250 gf, and further such as from 100 gf to 200 gf.

This hardness is measured in one of two ways. A first test for hardness is according to a method of penetrating a probe into the composition and in particular using a texture analyzer (for example TA-XT2i from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness is the “cheese wire” method, which involves cutting an 8.1 mm or preferably 12.7 mm [[NEW NUMBERS FROM FM???]] in diameter stick composition and measuring its hardness at 20° C using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 gf to 300 gf, such as from 30 gf to 250 gf, for a sample of 8.1 mm in diameter stick, and further such as from 30 gf to 200 gf, and also further such as from 30 gf to 120 gf for a sample of 12.7 mm in diameter stick.

The hardness of the composition of the present invention may be such that the compositions are self-supporting and can easily disintegrate to form a satisfactory deposit on keratin materials. In addition, this hardness may impart good impact strength to the inventive compositions, which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within preferred embodiments of the invention.

As is evident, the hardness of the composition according to preferred embodiments of the invention may, for example, be such that the composition is advantageously self-supporting and can disintegrate easily to form a satisfactory deposit on keratin materials. In addition, with this hardness, the composition of the invention may have good impact strength.

According to preferred embodiments of the present invention, the composition in stick form may have the behavior of a deformable, flexible elastic solid, giving noteworthy elastic softness on application.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion, bending or leaning if the composition is in stick form, melting, or syneresis (or sweating). The stability is further tested by repeating the 8-week test at 25° C., 37° C., 45° C. and under freeze-thaw conditions. A composition is considered to lack stability if in any of these tests an abnormality that impedes functioning of the composition is observed. The skilled artisan will readily recognize an abnormality that impedes functioning of a composition based on the intended application.

Silicone Elastomer

According to the present invention, compositions comprising at least one silicone elastomer are provided. Any suitable silicone elastomer can be used in accordance with the present invention. Suitable silicone elastomers include, for example, emulsifying silicone elastomers such as polyglycerolated and/or hydrophilic emulsifying silicone elastomers such as alkoxylated silicone elastomers, and non-emulsifying silicone elastomers. Such silicone elastomers can be spherical or non-spherical.

Preferably, the silicone elastomer is a “gellable elastomer.” A gellable elastomer is an elastomer capable of forming a gel when mixed with the appropriate solvent (for example, isododecane, dimethicone, cyclomethicone) and/or additional gelling agent(s). Some gellable elastomers useful in accordance with the present invention are sold commercially in a pre-gelled form. Others exist as particulate or powder. Either type of commercially available elastomer product may be used so long as the resulting material is gelled and/or capable of being gelled.

Polyglycerolated Silicone Elastomers

Suitable polyglycerolated silicone elastomers include, for example, crosslinked elastomeric organopolysiloxanes that may be obtained by a crosslinking addition reaction of diorganopolysiloxane containing at least one hydrogen atom linked to silicon and of polyglycerolated compounds containing ethylenically unsaturated groups, especially in the presence of a platinum catalyst.

Preferably, the crosslinked elastomeric organopolysiloxane is obtained by a crosslinking addition reaction (A) of diorganopolysiloxane containing at least two hydrogens each linked to a silicone, and (B) of glycerolated compounds containing at least two ethylenically unsaturated groups, especially in the presence (C) of a platinum catalyst.

In particular, the organopolysiloxane may be obtained by reaction of a polyglycerolated compound containing dimethylvinylsiloxy end groups and of methylhydrogenopolysiloxane containing trimethylsiloxy end groups, in the presence of a platinum catalyst.

Compound (A) is the base reagent for the formation of elastomeric organopolysiloxane and the crosslinking is performed by an addition reaction of compound (A) with compound (B) in the presence of the catalyst (C).

Compound (A) is in particular an organopolysiloxane containing at least 2 hydrogen atoms linked to different silicon atoms in each molecule.

Compound (A) may have any molecular structure, in particular a linear chain or branched chain structure or a cyclic structure.

Compound (A) may have a viscosity at 25° C. ranging from 1 to 50,000 centistokes, especially in order to have good miscibility with compound (B).

The organic groups linked to silicon atoms of the compound (A) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group. The said organic group is preferably chosen from methyl, phenyl and lauryl groups.

Compound (A) may thus be chosen from methylhydrogenopolysiloxanes containing trimethylsiloxy end groups, dimethylsiloxane-methylhydrogenosiloxane copolymers containing trimethylsiloxy end groups, dimethylsiloxane-methylhydrogenosiloxane cyclic copolymers, or dimethylsiloxane-methylhydrogenosiloxane-laurylmethylsiloxane copolymers containing trimethylsiloxy end groups.

Compound (B) may be a polyglycerolated compound corresponding to formula (B′) below:

CₘH₂ₘ₋₁—O-[Gly]ₙ-CₘH₂ₘ₋₁ (B′)

in which m is an integer ranging from 2 to 6, n is an integer ranging from 2 to 200, preferably ranging from 2 to 100, preferably ranging from 2 to 50, preferably ranging from 2 to 20, preferably ranging from 2 to 10 and preferably ranging from 2 to 5, and in particular equal to 3; Gly denotes:

—CH₂—CH(OH)—CH₂—O— or —CH₂—CH(CH₂OH)—O—

Advantageously, the sum of the number of ethylenic groups per molecule of compound (B) and of the number of hydrogen atoms linked to silicon atoms per molecule of compound (A) is at least 4.

It is advantageous for compound (A) to be added in an amount such that the molar ratio between the total amount of hydrogen atoms linked to silicon atoms in compound (A) and the total amount of all the ethylenically unsaturated groups in compound (B) is within the range from 1/1 to 20/1.

Compound (C) is the crosslinking reaction catalyst, and is especially chloroplatinic acid, chloroplatinic acid-olefin complexes, chloroplatinic acid-alkenylsiloxane complexes, chloroplatinic acid-diketone complexes, platinum black and platinum on a support.

The catalyst (C) is preferably added in from 0.1 to 1,000 parts by weight, better still from 1 to 100 parts by weight, as clean platinum metal per 1,000 parts by weight of the total amount of compounds (A) and (B).

The polyglycerolated silicone elastomer may be conveyed in gel form in at least one hydrocarbon-based oil and/or one silicone oil.

Polyglycerolated silicone elastomers that may be used include, but are not limited to, those sold under the names “KSG-710”, “KSG-810”, “KSG-820”, “KSG-830” and “KSG-840” by the company Shin-Etsu. Suitable polygycerolated silicone elastomers are also disclosed in U.S. Ser. No. 11/085,509, filed Mar. 22, 2005 (published as U.S. patent application publication no. 2005/0220728), the entire disclosure of which is hereby incorporated by reference.

Hydrophilic Emulsifying Silicone Elastomers

The term “hydrophilic emulsifying silicone elastomer” means a silicone elastomer comprising at least one hydrophilic chain other than a polyglycerolated chain as described above.

In particular, the hydrophilic emulsifying silicone elastomer may be chosen from polyoxyalkylenated silicone elastomers.

Suitable polyoxyalkylenated silicone elastomers include crosslinked organopolysiloxanes that may be obtained by a crosslinking addition reaction of diorganopolysiloxane containing at least one hydrogen linked to silicon and of a polyoxyalkylene containing at least two ethylenically unsaturated groups.

Preferably, the polyoxyalkylenated crosslinked organopolysiloxane is obtained by a crosslinking addition reaction (A1) of diorganopolysiloxane containing at least two hydrogens each linked to a silicon, and (B1) of polyoxyalkylene containing at least two ethylenically unsaturated groups, especially in the presence (C1) of a platinum catalyst, as described, for example, in patents U.S. Pat. No. 5,236,986 and U.S. Pat. No. 5,412,004, the entire disclosures of which are hereby incorporated by reference.

The organopolysiloxane may be obtained by reaction of polyoxyalkylene (especially polyoxyethylene and/or polyoxypropylene) containing dimethylvinylsiloxy end groups and of methylhydrogenopolysiloxane containing trimethylsiloxy end groups, in the presence of a platinum catalyst.

The organic groups linked to silicon atoms of the compound (A1) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

Compound (A1) may thus be chosen from methylhydrogenopolysiloxanes containing trimethylsiloxy end groups, dimethylsiloxane-methylhydrogenosiloxane copolymers containing trimethylsiloxy end groups, dimethylsiloxane-methylhydrogenosiloxane cyclic copolymers, dimethylsiloxane-methylhydrogenosiloxane-laurylmethylsiloxane copolymers containing trimethylsiloxy end groups.

Compound (C1) is the crosslinking reaction catalyst, and is especially chloroplatinic acid, chloroplatinic acid-olefin complexes, chloroplatinic acid-alkenylsiloxane complexes, chloroplatinic acid-diketone complexes, platinum black and platinum on a support.

Preferably, the polyoxyalkylenated silicone elastomers may be formed from divinyl compounds, in particular polyoxyalkylenes containing at least two vinyl groups, reacting with Si—H bonds of a polysiloxane.

The polyoxyalkylenated silicone elastomers may be conveyed in the form of a gel consisting of an elastomeric organopolysiloxane included in at least one hydrocarbon-based oil and/or one silicone oil.

Suitable polyoxyalkylenated elastomers are described in patents U.S. Pat. No. 5,236,986, U.S. Pat. No. 5,412,004, U.S. Pat. No. 5,837,793 and U.S. Pat. No. 5,811,487, the entire contents of which are incorporated herein by reference.

Suitable polyoxyalkylenated silicone elastomers that may be used include those sold under the names “KSG-21”, “KSG-20”, “KSG-30”, “KSG-31”, “KSG-32”, “KSG-33”, “KSG-210”, “KSG-310”, “KSG-320”, “KSG-330”, “KSG-340” and “X-226146” by the company Shin-Etsu, or “DC9010” and “DC9011” by the company Dow Corning.

Suitable hydrophilic emulsifying silicone elastomers are also disclosed in U.S. Ser. No. 11/085,509, filed Mar. 22, 2005 (published as U.S. patent application publication no. 2005/0220728), the entire disclosure of which is hereby incorporated by reference.

Non-Emulsifying Silicone Elastomers

The term “non-emulsifying” defines elastomers not containing a hydrophilic chain, such as polyoxyalkylene or polyglycerolated units.

The non-emulsifying silicone elastomer is preferably an elastomeric crosslinked organopolysiloxane that may be obtained by a crosslinking addition reaction of diorganopolysiloxane containing at least one hydrogen linked to silicon and of diorganopolysiloxane containing ethylenically unsaturated groups linked to silicon, especially in the presence of a platinum catalyst; or by dehydrogenation crosslinking coupling reaction between a diorganopolysiloxane containing hydroxyl end groups and a diorganopolysiloxane containing at least one hydrogen linked to silicon, especially in the presence of an organotin compound; or by a crosslinking coupling reaction of a diorganopolysiloxane containing hydroxyl end groups and of a hydrolysable organopolysilane; or by thermal crosslinking of organopolysiloxane, especially in the presence of an organoperoxide catalyst; or by crosslinking of organopolysiloxane via high-energy radiation such as gamma rays, ultraviolet rays or an electron beam.

Preferably, the elastomeric crosslinked organopolysiloxane is obtained by a crosslinking addition reaction (A2) of diorganopolysiloxane containing at least two hydrogens each linked to a silicon, and (B2) of diorganopolysiloxane containing at least two ethylenically unsaturated groups linked to silicon, especially in the presence (C2) of a platinum catalyst, as described, for example, in patent application EP0295886A, the entire disclosure of which is hereby incorporated by reference.

The organopolysiloxane may be obtained by reaction of dimethylpolysiloxane containing dimethylvinylsiloxy end groups and of methylhydrogenopolysiloxane containing trimethylsiloxy end groups, in the presence of a platinum catalyst.

Compound (A2) is the base reagent for the formation of elastomeric organopolysiloxane, and the crosslinking is performed by an addition reaction of compound (A2) with compound (B2) in the presence of a catalyst (C2).

Compound (A2) is advantageously a diorganopolysiloxane containing at least two lower (for example C2-C4) alkenyl groups; the lower alkenyl group may be chosen from vinyl, allyl and propenyl groups. These lower alkenyl groups may be located in any position on the organopolysiloxane molecule, but are preferably located at the ends of the organopolysiloxane molecule. The organopolysiloxane (A2) may have a branched-chain, linear-chain, cyclic or network structure, but the linear-chain structure is preferred. Compound (A2) may have a viscosity ranging from the liquid state to the gum state. Preferably, compound (A2) has a viscosity of at least 100 centistokes at 25° C.

The organopolysiloxanes (A2) may be chosen from methylvinylsiloxanes, methylvinylsiloxane-dimethylsiloxane copolymers, dimethylpolysiloxanes containing dimethylvinylsiloxy end groups, dimethylsiloxane-methylphenylsiloxane copolymers containing dimethylvinylsiloxy end groups, dimethylsiloxane-diphenylsiloxane-methylvinylsiloxane copolymers containing dimethylvinylsiloxy end groups, dimethylsiloxane-methylvinylsiloxane copolymers containing trimethylsiloxy end groups, dimethylsiloxane-methylphenylsiloxane-methylvinylsiloxane copolymers containing trimethylsiloxy end groups, methyl(3,3,3-trifluoropropyl)polysiloxanes containing dimethylvinylsiloxy end groups, and dimethylsiloxane-methyl(3,3,3-trifluoropropyl)siloxane copolymers containing dimethylvinylsiloxy end groups.

Compound (B2) is in particular an organopolysiloxane containing at least 2 hydrogens linked to silicon in each molecule and is thus the crosslinking agent for the compound (A2).

Preferably, the sum of the number of ethylenic groups per molecule of compound (A2) and the number of hydrogen atoms linked to silicon per molecule of compound (B2) is at least 4.

Compound (B2) may be in any molecular structure, especially of linear-chain or branched-chain structure, or cyclic structure.

Compound (B2) may have a viscosity at 25° C. ranging from 1 to 50,000 centistokes, especially in order to have good miscibility with compound (A).

It is advantageous for compound (B2) to be added in an amount such that the molar ratio between the total amount of hydrogen atoms linked to silicon in compound (B2) and the total amount of all of the ethylenically unsaturated groups in compound (A2) is within the range from 1/1 to 20/1.

Compound (B2) may be chosen from methylhydrogenopolysiloxanes containing trimethylsiloxy end groups, dimethylsiloxane-methylhydrogenosiloxane copolymers containing trimethylsiloxy end groups, and dimethylsiloxane-methylhydrogenosiloxane cyclic copolymers.

Compound (C2) is the crosslinking reaction catalyst, and is especially chloroplatinic acid, chloroplatinic acid-olefin complexes, chloroplatinic acid-alkenylsiloxane complexes, chloroplatinic acid-diketone complexes, platinum black and platinum on a support.

The catalyst (C2) is preferably added in from 0.1 to 1,000 parts by weight, better still from 1 to 100 parts by weight, as clean platinum metal per 1,000 parts by weight of the total amount of compounds (A2) and (B2).

Other organic groups may be linked to silicon in the organopolysiloxanes (A2) and (B2) described above, for instance alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

The elastomeric crosslinked organopolysiloxanes may be conveyed in the form of a gel consisting of an elastomeric organopolysiloxane included in at least one hydrocarbon-based oil and/or one silicone oil. The elastomeric crosslinked organopolysiloxanes may also be in powder form.

Suitable non-emulsifying silicone elastomers are described in patent applications JP61-194009 A, EP0242219 A, EP0295886 A and EP0765656 A, the entire contents of which are herein incorporated by reference.

Suitable non-emulsifying silicone elastomers that may be used include, but are not limited to, those sold under the names “DC 9040”, “DC 9041”, “DC 9509”, “DC 9505” and “DC 9506” by the company Dow Corning.

Suitable non-emulsifying silicone elastomers are also disclosed in U.S. Ser. No. 11/085,509, filed Mar. 22, 2005 (published as U.S. patent application publication no. 2005/0220728), the entire disclosure of which is hereby incorporated by reference.

The non-emulsifying silicone elastomer may also be in the form of elastomeric crosslinked organopolysiloxane powder coated with silicone resin, especially with silsesquioxane resin, as described, for example, in patent U.S. Pat. No. 5,538,793, the entire content of which is herein incorporated by reference. Such elastomers are sold under the names “KSP-100”, “KSP-101” “KSP-102”, “KSP-103”, “KSP-104” and “KSP-105” by the company Shin-Etsu.

Other elastomeric crosslinked organopolysiloxanes in the form of powders include hybrid silicone powders functionalized with fluoroalkyl groups, sold especially under the name “KSP-200” by the company Shin-Etsu; hybrid silicone powders functionalized with phenyl groups, sold especially under the name “KSP-300” by the company Shin-Etsu.

Preferably, the at least one elastomer is present in an amount ranging from about 0.1% to about 25% by weight of active material with respect to the total weight of the composition, more preferably from about 0.2% to about 20%, more preferably from about 0.25% to about 15%, and most preferably from about 0.5% to about 10% by weight of active material with respect to the total weight of the composition, including all ranges and subranges therebetween.

Oil

According to the present invention, compositions comprising at least one oil are provided. Any oil(s) can be used in accordance with the present invention. The oils can be volatile or non-volatile, silicone-based and/or hydrocarbon-based, etc. Thus, for example, the oil phase may contain, independently or in combination, volatile silicone oils, non-volatile silicone oils, volatile non-silicone oils and non-volatile non-silicone oils.

In one embodiment, the compositions of the present invention are substantially free of silicone oils (i.e., contain less than about 5% of silicone oil). In another embodiment, the compositions are substantially free of non-volatile oils (i.e., contain less than about 5% of non-volatile oil). In yet another embodiment, the compositions are substantially free of volatile oils (i.e., contain less than about 5% of volatile oil). According to such embodiments, the compositions may also be essentially free of the types of oils indicated above (i.e., contain less than about 3% of the oil) or free of the types of oils indicated above (i.e., contain less than about 1% of the oil).

According to particularly preferred embodiments of the present invention, the amount of non-silicone oil present is equal to or greater than the amount of silicone oil present in the compositions of the present invention. That is, the ratio of non-silicone oil to silicone oil is at least 1:1, more preferably 2:1, more preferably 3:1, more preferably 5:1, more preferably 10:1, etc. According to preferred embodiments, more non-silicone oil is present in the compositions of the present invention than silicone oil.

According to one embodiment, the oil phase may contain one or more volatile silicone oils. Examples of such volatile silicone oils include linear or cyclic silicone oils having a viscosity at room temperature less than or equal to 6 cSt and having from 2 to 7 silicone atoms, these silicones being optionally substituted with alkyl or alkoxy groups of 1 to 10 carbon atoms. Suitable oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane (L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3St) from Dow Corning | 102 | 3 |

Further, a volatile linear silicone oil may be employed in the compositions of the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

The volatility of the solvents/oils can be determined using the evaporation speed as set forth in U.S. Pat. No. 6,338,839.

Examples of other silicone oils that may be used in the invention include non-volatile linear polydimethylsiloxanes (PDMSs), that are liquid at room temperature; polydimethylsiloxanes comprising alkyl, alkoxy or phenyl groups, which are pendent and/or at the end of a silicone chain, these groups each containing from 2 to 24 carbon atoms; phenylsilicones, for instance phenyl trimethicones, phenyl dimethicones, phenyl trimethylsiloxydiphenylsiloxanes, diphenyl dimethicones, diphenyl methyidiphenyl trisiloxanes and 2-phenylethyl trimethylsiloxysilicates.

According to other preferred embodiments, the oil phase may contain one or more non-silicone volatile oils and may be selected from volatile hydrocarbon oils, alcohols, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate and their mixtures. Preferably, the volatile non-silicone oils have a flash point of at least 40° C.

TABLE 2

| Compound | Flash Point (° C.) |
|---|---|
| Isododecane | 43 |
| Isohexadecane | 102 |
| Isodecyl Neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

Examples of other non-silicone oils which can be used in the compositions of the present invention include polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R₅ represents a linear or branched higher fatty acid residue containing from 1 to 40 carbon atoms, including and better still from 7 to 19 carbon atoms, and R₆ represents a branched hydrocarbon-based chain containing from 1 to 40 carbon atoms, including and better still from 3 to 20 carbon atoms, with R₆+R₇≧10, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C₈ to C₂₆ fatty alcohols, for instance oleyl alcohol; and
mixtures thereof.

Preferably, the oil(s) represent from about 25% to about 70% by weight of the total weight of the composition, more preferably from about 30% to about 60% of the total weight of the composition, more preferably from about 35% to about 55% of the total weight of the composition, and most preferably from about 40% to about 50%, including all ranges and subranges therebetween.

Wax

According to the present invention, compositions capable of imparting a satin-to-matte appearance to keratin materials such as lips or skin comprising at least one wax are provided. For the purposes of the present invention, a wax is a lipophilic fatty compound that is solid at room temperature (25° C.) and atmospheric pressure (760 mmHg, i.e. 101 KPa), which undergoes a reversible solid/liquid change of state, having a melting point of greater than 40° C. and further such as greater than 55° C. and which may be up to 200° C., where the melting point values correspond to the melting peak measured by the “Differential Scanning Calorimetry” method with a temperature rise of 5 or 10° C./min. By bringing the wax to its melting point, it is possible to make it miscible with oils and to form a microscopically homogeneous mixture, but on returning the temperature of the mixture to room temperature, recrystallization of the wax in the oils of the mixture is obtained.

Suitable waxes include those generally used in cosmetics and dermatology such as, for example, waxes of natural origin, for instance beeswax, ozokerite, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., fatty alcohol waxes such as those sold by Baker Petrolite under the Performacol name (Performacol 350, 425 and 550) including C30-C50 alcohols, silicone waxes such as alkyl- and alkoxy-poly(di)methylsiloxanes and/or poly(di)methyl-siloxane esters that are solid at 40° C., for example, at above 55° C.

Preferably, the at least one wax is present in an amount ranging from about 0.5% to about 50% by weight of active material with respect to the total weight of the composition, more preferably from about 2% to about 40%, more preferably from about 5% to about 30%, more preferably from about 2% to about 40%, more preferably from about 8% to about 25%, and most preferably from about 10% to about 20% by weight of active material with respect to the total weight of the composition, including all ranges and subranges therebetween.

Coloring Agents

According to the present invention, the compositions further comprise at least one coloring agent (colorant). Suitable coloring agents include but are not limited to pigments, dyes, such as liposoluble dyes, nacreous pigments, and pearling agents. Typically, when the composition contains substantial amounts of colorants, it is a make-up composition such as a lipstick or foundation. Alternatively, when the composition does not contain a significant amount of colorants, it can be clear or transparent and can be used as a basecoat (or topcoat) prior to (or after) application of a make-up composition, or it can be used as a hair treatment composition such as, for example, a hair conditioner or mousse. However, it is possible that topcoats, basecoats, hair treatment products and the like could contain colorants, and/or that a make-up composition could contain little colorant.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.1% to 20%, preferably from 0.1% to 15%.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 40% by weight of the total weight of the composition, such as from 0,5% to 30%, from 1% to 25%, and from 2% to 20%, including all ranges and subranges therebetween. In the case of certain products, the pigments, including nacreous pigments, may, for example, represent up to 50% by weight of the composition.

Filler

According to the present invention, compositions capable of imparting a satin-to-matte appearance to keratin materials such as lips or skin comprising at least one filler are provided.

Acceptable fillers include, but are not limited to, inorganic and organic fillers which may have any shape such as, for example, lamellar, spherical and/or oblong. Non-limiting examples of suitable fillers include, but are not limited to, polyamide powders (such as Nylon® powder, and such as the product sold by Atochem as Orgasol®)), poly-β-alanine powders, polyethylene powders, acrylic polymer powders (such as polymethyl methacrylate (PMMA) powder, for instance the product sold by Wacker as Covabead LH-85 (particle size 10-12 μm) and the acrylic acid copolymer powder sold by Dow Corning as Polytrap®), polytetrafluoroethylene (Teflon®) powders, lauroyllysine, boron nitride, starch, starch derivatives, hollow polymer microspheres (such as those hollow polymer microspheres formed from polyvinylidene chloride and acrylonitrile, for instance the product sold by Nobel Industrie as Expancel®), and polymerized silicone microspheres (such as those polymerized silicone microspheres sold by Toshiba as Tospearl®), precipitated calcium carbonate, magnesium carbonate and hydrocarbonate, hydroxyapatite, hollow silica microspheres (such as the product sold by Maprecos as Silica Beads®), glass microcapsules, ceramic microcapsules, and polyester particles.

Preferably, the at least one filler is present in an amount ranging from about 0.5% to about 50% by weight of active material with respect to the total weight of the composition, more preferably from about 1% to about 30%, more preferably from about 3% to about 25%, and most preferably from about 4% to about 20% by weight of active material with respect to the total weight of the composition, including all ranges and subranges therebetween.

According to particularly preferred embodiments, the compositions contain so little clay material filler (kaolin, talc, bentonite) that the presence of such clay materials does not adversely affect the cosmetic properties of the composition such as, for example, the drag, feel, and/or opaqueness of the composition. Preferably, the compositions are substantially free of such clay material fillers (i.e., contain less than about 0.5% clay materials), essentially free of such clay material fillers (i.e., contain less than about 0.25% clay material fillers) or free of such clay material fillers (i.e., contain less than about 0.1% clay material fillers).

According to other particularly preferred embodiments, the combined total amount of silicone elastomer(s), wax(es), filler(s) and coloring agent(s) present is at least about 30% by weight with respect to the total weight of the composition, more preferably at least about 35%, more preferably at least about 40%, and more preferably at least about 45%, including all ranges and subranges therebetween.

According to other preferred embodiments, the ratio of the total amount of oil present in the composition to the combined total amount of silicone elastomer(s), wax(es), filler(s) and coloring agent(s) present in the composition ranges from about 3:1 to about 1:3, preferably from about 2.5:1 to about 1:2.5, and more preferably from about 1.5: 1 to about 1:1.5, including all ranges and subranges therebetween.

Film Forming/Structuring Agents

According to particularly preferred embodiments of the present invention, compositions further comprising at least one at least one film forming or structuring polymer such as, for example, a copolymer comprising at least one styrene block, a hydrocarbon resin and/or a polyorganosiloxane-containing polymer, are provided.

Acceptable film forming agents are known in the art and include, but are not limited to, those disclosed in U.S. patent application publication no. 2004/0170586, the entire contents of which is hereby incorporated by reference. Non-limiting representative examples of such film forming agents include silicone resins such as, for example, MQ resins (for example, trimethylsiloxysilicates) and MK resins (for example, polymethylsilsesquioxanes), silicone esters such as those disclosed in U.S. Pat. Nos. 6,045,782, 5,334,737, and 4,725,658, the disclosures of which are hereby incorporated by reference, polymers comprising a backbone chosen from vinyl polymers, methacrylic polymers, and acrylic polymers and at least one chain chosen from pendant siloxane groups and pendant fluorochemical groups such as those disclosed in U.S. Pat. Nos. 5,209,924, 4,693,935, 4,981,903, 4,981,902, and 4,972,037, and WO 01/32737, the disclosures of which are hereby incorporated by reference, polymers such as those described in U.S. Pat. No. 5,468,477, the disclosure of which is hereby incorporated by reference (a non-limiting example of such polymers is poly(dimethylsiloxane)-g-poly(isobutyl methacrylate), which is commercially available from 3M Company under the tradename VS 70 IBM).

Suitable copolymers comprising at least one styrene block include, but are not limited to, triblock copolymers such as those of the polystyrene/polyisoprene or polystyrene/polybutadiene type, for example those sold or made under the name “Luvitol HSB” by BASF, and those of the polystyrene/copoly(ethylene-propylene) type or alternatively of the polystyrene/copoly(ethylene/butylene) type, such as those sold or made under the brand name “Kraton” by Shell Chemical Co. or Gelled Permethyl 99A by Penreco. Specific examples include Kraton (G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton G1750X (EP) multiarm, Kraton G1765X (EP) multiarm, Kraton D-1101 (SBS), Kraton D-1102 (SBS), Kraton D-1107 (SIS), Gelled Permethyl 99A-750, Gelled Permethyl 99A-753-58 (mixture of starburst block polymer and triblock polymer), Gelled Permethyl 99A-753-59 (mixture of starburst block polymer and triblock polymer), Versagel 5970 and Versagel 5960 from Penreco (mixture of starburst polymer and triblock polymer in isododecane), and OS 129880, OS 129881 and OS 84383 from Lubrizol (styrene-methacrylate copolymer). Also, such polymers are described in patent applications WO 98/38981 and US 2002/0055562, the disclosures of which are hereby incorporated by reference.

Suitable hydrocarbon resins include but are not limited to aliphatic hydrocarbon resins, hydrogenated aliphatic hydrocarbon resins, aliphatic/aromatic hydrocarbon resins, hydrogenated aliphatic aromatic hydrocarbon resins, cycloaliphatic hydrocarbon resins, hydrogenated cycloaliphatic resins, cycloaliphatic/aromatic hydrocarbon resins, hydrogenated cycloaliphatic/aromatic hydrocarbon resins, aromatic hydrocarbon resins, hydrogenated aromatic hydrocarbon resins, polyterpene resins, terpene-phenol resins, rosins, rosin esters, resins grafted with an unsaturated acid or anhydride, and mixtures of any two or more thereof. When referring to hydrogenated resins, hydrogenated includes resins that are at least partially hydrogenated and substantially hydrogenated.

Examples of suitable hydrocarbon resins include but are note limited to ESCOREZ™ 1310 and EMPR™ 118 available from ExxonMobil Chemical Company, Houston, Tex., PICCOTAC™ 1020, 1020E, and 9095 available from Eastman Chemical Company, Kingsport, Tenn., WINGTACK™ 10, 86, PLUS, and 95 available from Goodyear Chemical Company, and QUINTONE™ K100, R100, and M100 available from Nippon Zeon of Japan.

Other suitable hydrocarbon resins are disclosed in U.S. patent application publication no. 2004/0092648, published May 13, 2004, the entire contents of which is hereby incorporated by reference.

Suitable hydrocarbon resins further include low molecular weight, lightly colored, inert thermoplastic resins derived from petrochemical feedstocks. Preferably, these thermoplastic polymers are also partially or fully hydrogenated. These include certain hydrogenated polycyclopentadienes and hydrogenated styrene/methylstyrene/indene copolymers sold under the trade name REGALITE. Some of the REGALITES are made from C₈₊ monomers which include, without limitation, vinyl toluene, dicyclopentadiene, indene, alpha-methyl styrene, styrene and methyl indene. These low molecular weight hydrocarbon resins may be found in a number of commercial products including without limitation those sold by Eastman Chemical Middelburg BV, Tobias Asserlaan 5, 2517 KC Den Haag, the Netherlands, under the trademark REGALITE, PICCOTAC and EASTOTAC. A material that typifies a hydrocarbon resin that may be used in accordance with the present invention is REGALITE® R1090 hydrogenated thermoplastic resin, as described in product data sheet 65.014-E3, dated February, 2001. Other useful polyolefins of this type include REGALITE® R1125, R1100 and R9100.

Suitable polyorganosiloxane-containing polymers can generally be described as polymers chosen from homopolymers and copolymers, preferably, with a weight-average molecular mass ranging from about 500 to about 2.5×10⁶ or more, comprising at least one moiety comprising: at least one polyorganosiloxane group comprising, preferably, from 1 to about 10,000 organosiloxane units in the chain of the moiety or in the form of a graft, and at least two groups capable of establishing hydrogen interactions. Examples of suitable polyorganosiloxane-containing polymers can be found in U.S. patent application Ser. No. 11/254,919, filed Oct. 21, 2005, the entire contents of which is hereby incorporated by reference.

More specifically, preferred polyorganosiloxane-containing polymers comprise at least one moiety chosen from formulae (III) and (IV):

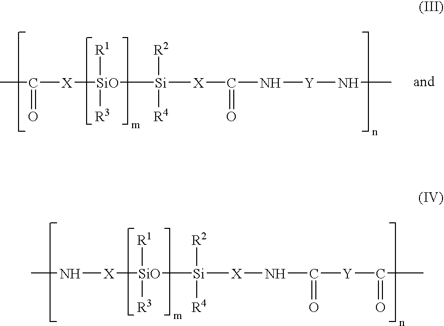

in which:
1) R¹, R², R³ and R⁴, which may be identical or different, are chosen from:
   linear, branched and cyclic, saturated and unsaturated, C₁ to C₄₀ hydrocarbon-based groups, optionally comprising in the chain at least one atom chosen from oxygen, sulphur and nitrogen, and also optionally being partially or totally substituted with at least one fluorine atom,
   C₆ to C₁₀ aryl groups, optionally substituted with at least one C₁ to C₄ alkyl group,
   polyorganosiloxane chains optionally comprising at least one atom chosen from oxygen, sulphur and nitrogen;
2) the groups X, which may be identical or different, are chosen from linear and branched C₁ to C₃₀ alkylenediyl groups, optionally comprising in the chain at least one atom chosen from oxygen and nitrogen;
3) Y is chosen from saturated and unsaturated, C₁ to C₅₀ linear and branched divalent alkylene, arylene, cycloalkylene, alkylarylene and arylalkylene groups, optionally comprising at least one atom chosen from oxygen, sulphur and nitrogen, and optionally substituted by one of the following atoms and groups of atoms:
   fluorine, hydroxyl, C₃ to C₈ cycloalkyl, C₁ to C₄₀ alkyl, C₅ to C₁₀ aryl, phenyl optionally substituted with 1 to 3 C₁ to C₃ alkyl groups, C₁ to C₃ hydroxyalkyl and C₁ to C₆ aminoalkyl, or
4) Y represents a group corresponding to the formula:

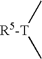

in which
T is chosen from linear and branched, saturated and unsaturated, C₃ to C₂₄ trivalent and tetravalent hydrocarbon-based groups optionally substituted with a polyorganosiloxane chain, and optionally comprising at least one atom chosen from O, N and S, or T represents a trivalent atom chosen from N, P and Al, and
R⁵ is chosen from linear and branched C₁ to C₅₀ alkyl groups and polyorganosiloxane chains, optionally comprising at least one group chosen from ester, amide, urethane, thiocarbamate, urea, thiourea and sulphonamide groups, which may optionally be linked to another chain of the polymer;
5) n is an integer ranging from 2 to 500, and m is an integer ranging from 1 to 1 000.

Particularly preferred polyorganosiloxane-containing polymers are polysiloxane-polyamide copolymers available from Dow Corning such as, for example, Nylon-611/Dimethicone copolymer.

According to preferred embodiments, the film former/structuring agent, when present, is present in the composition in an amount ranging from 0.1% to 30% by weight relative to the total weight of the composition. Preferably, the film former is present in an amount ranging from 0.5% to 20% by weight relative to the total weight of the composition, more preferably from 0.75% to 10%, more preferably from 1% to 8%, and more preferably from 2% to 6%, including all ranges and subranges therebetween. One of ordinary skill in the art will recognize that the film former/structuring agent of the present invention may be commercially available, and may come from suppliers in the form of a dilute solution. The amounts of the film former disclosed herein therefore reflect the weight percent of active material.

According to particularly preferred embodiments of the present invention, the compositions of the present invention are anhydrous. Preferably, the compositions are substantially anhydrous or substantially free of water (i.e., contain less than about 3% water), essentially anhydrous or essentially free of water (i.e., contain less than about 1% water) or anhydrous or free of water (i.e., contain less than about 0.5% water).

According to other embodiments, the compositions of the present invention further comprise water. In this embodiment, water is preferably present in an amount ranging from about 0.6 to about 70%, preferably from about 3.0 to 60%, and more preferably from about 5 to about 50% relative to the total weight of the composition.

Additional Additives

The composition of the invention can also comprise any additive usually used in the field under consideration. For example, dispersants such as poly(12-hydroxystearic acid), antioxidants, essential oils, sunscreens, preserving agents, fragrances, neutralizing agents, cosmetic and dermatological active agents such as, for example, moisturizers, vitamins, essential fatty acids, surfactants and mixtures thereof can be added. A non-exhaustive listing of such ingredients can be found in U.S. patent application publication no. 2004/0170586, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the keratin materials of human beings.

According to preferred embodiments of the present invention, methods of producing a composition capable of imparting a satin-to-matte appearance to keratin materials, preferably a solid composition, comprising combining at least one oil, at least one silicone elastomer, at least one wax, at least one filler and at least one coloring agent to form a composition are provided. According to particularly preferred embodiments, these methods include combining ingredients to form a composition which is substantially free of, essentially free of or free of clay materials such as talc, kaolin and bentonite.

According to preferred embodiments of the present invention, methods of minimizing drag or cakey feeling associated with application of compositions which impart a satin-to-matte appearance to keratin materials comprising combining at least one oil, at least one silicone elastomer, at least one wax, at least one filler, and at least one coloring agent to form a composition capable of imparting a satin-to-matte appearance to keratin materials are provided.

According to other preferred embodiments of the present invention, methods of delivering a satin-to-matte appearance to a keratin material comprising applying a composition comprising at least one oil, at least one silicone elastomer, at least one wax, at least one filler, and at least one coloring agent to the keratin material in an amount sufficient to deliver a satin-to-matte appearance to a keratin material are provided.

According to yet other preferred embodiments of the present invention, methods of minimizing bleeding, feathering or oil migration upon application to a keratin material of a cosmetic composition comprising at least about 30% oil comprising combining at least 30% oil, at least one silicone elastomer, at least one wax, at least one filler, and at least one coloring agent to form a composition are provided.

According to preferred embodiments of the present invention, methods of treating or caring for keratin materials by applying compositions of the present invention to the keratin materials in an amount sufficient to treat and/or care for the keratin materials are provided.

According to yet other preferred embodiments of the present invention, methods of enhancing the appearance of and/or making up keratin materials by applying compositions of the present invention to the keratin materials in an amount sufficient to enhance the appearance of the keratin materials are provided. Preferably, “making up” the keratin material includes applying at least one coloring agent to the keratin material in an amount sufficient to provide color to the keratin material.

In accordance with the three preceding embodiments, the compositions of the present invention are applied topically to the desired area of keratin material (skin or lips) in an amount sufficient to deliver a satin-to-matte appearance to, treat, care for, enhance the appearance of and/or make up the keratinous material. The compositions may be applied to the desired area as needed, preferably once or twice daily, more preferably once daily, and then preferably allowed to dry before subjecting to contact such as with clothing or other objects (for example, a glass or a topcoat). Preferably, the composition is allowed to dry for about 1 minute or less. The composition is preferably applied to the desired area that is dry or has been dried prior to application, or to which a basecoat has been previously applied.

The present invention also envisages kits and/or prepackaged materials suitable for consumer use containing one or more compositions according to the description herein. The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

In accordance with this embodiment, the kit may comprise (a) a composition capable of imparting a satin-to-matte appearance to keratin materials (skin or lips) comprising at least one oil, at least one silicone elastomer, at least one wax, at least one filler, and at least one coloring agent; and (b) a basecoat and/or topcoat composition. In accordance with this embodiment, the composition (a) corresponds to the invention compositions described above, while the composition (b) is/are composition(s) applied before and/or after the invention composition, typically known in the art as basecoats (applied before) and topcoats (applied after). No particular requirements exist for the basecoats and topcoats which can be used in conjunction with the compositions of the present invention and, thus, such basecoats and topcoats can contain ingredients such as those discussed above. Preferably, however, such basecoats and topcoats should not have overly deleterious effects on the properties of the invention compositions (for example, the basecoats or topcoats should not unacceptably affect the transfer-resistance properties of the invention compositions).

In a preferred embodiment, kits of the present invention comprise two or more solid compositions. For example, the kit may comprise (a) a solid composition capable of imparting a satin-to-matte appearance to keratin materials (skin or lips) comprising at least one oil, at least one silicone elastomer, at least one wax, at least one filler, and at least one coloring agent; and (b) a solid basecoat and/or topcoat composition.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### I. COMPOSITIONS

### EXAMPLE A

### Lip Mousse Pot

EXAMPLE A
Lip Mousse Pot

| Phase | INCI names | % wt/wt |
|---|---|---|
| A | phenyl trimethicone | 4.48% |
| | Hydrogenated Polyisobutene | 6.46% |
| | C12-15 alkyl benzoate | 1.20% |
| | Tridecyl Trimellitate | 1.79% |
| B | Pigments & Pearls | 13.00% |
| C | Bentone | 0.13% |
| | Micronized TiO2 | 1.00% |
| D | Octyldodecanol and Tocopherol Acetate and Silica | 2.40% |
| | Tridecyl Trimellitate | 6.92% |
| | shea butter | 0.40% |
| | phenyl trimethicone | 3.60% |
| | Dimethicone (5 cSt) | 12.60% |
| E | dimethicone-dimethicone elastomer (DC9041) | 6.30% |
| | Cyclopentasiloxane-Dimethicone Crosspolymer | 17.60% |
| | (DC5930) | |
| | dimethicone-vinyl dimethicone elastomer (dc9506) | 1.82% |
| F | silica-vinyl dimethicone powder | 3.00% |
| | methyl methacrYlate | 0.90% |
| | HDI/Trimethylol Hexyllactone Crosspolymer | 2.80% |
| | Vinyl Dimethicone-Methicone Silesquioxane | 1.00% |
| | crosspolymer (KSP100) | |
| G | Beeswax | 3.00% |
| | ozokerite wax | 3.60% |
| | C12-45 alkyl dimethicone | 1.80% |
| H | (70% Polycyclopentadiene in isododecane)) | 4.20% |
| | | 100.00% |

### EXAMPLE B

### Mousse Lip Stick

EXAMPLE B
Mousse Lip Stick

| Trade Name | INCI Name | % |
|---|---|---|
| Dry pigment | | 13.00% |
| pearls/mica | | |
| DC556 Fluid | phenyl trimethicone (QS liquid) | 5.26% |
| Polysynlane | Hydrogenated Polyisobutene | 13.35% |
| Tegosoft TN | C12-15 alkyl benzoate | 1.64% |
| Liponate TDTM | Tridecyl Trimellitate | 16.14% |
| Isostearyl | Isostearyl isostearate | 8.00% |
| isostearate | | |
| DC9041 | dimethicone-dimethicone elastomer | 11.20% |
| KSP 100 | vinyl dimethicone/methicone | 1.20% |
| | silsesquioxane crosspoloymer | |

| | |
|---|---|
| jojoba esters | 4.44% |
| ozokerite wax | 5.68% |
| polyethylene wax | 1.00% |
| silica-vinyl dimethicone powder | 2.00% |
| HDI/Trimethylol Hexyllactone Crosspolymer | 1.50% |
| Nylon-12 | 0.80% |
| Polycyclopentadiene | 4.40% |
| Octyldodecanol and Tocopherol Acetate | 1.00% |
| and Silica | |
| BHT | 0.05% |
| Aloe Vera Extract | 0.70% |
| Ultrafine Titanium Dioxide | 1.00% |
| Shea Butter | 0.20% |
| Fragrance | 0.20% |
| Gel phase | 6.84% |
| Bentone | 0.40% |
| | 100% |

### II. COMPARATIVE EXAMPLES

### Comparison of the Presence of Silicone and Non-Silicone Oils on Appearance

| | | | | Example D - |
|---|---|---|---|---|
| | | | | Comparative |
| | | Example C - | | Mousse stick |
| | | Mousse stick | | containing |
| | | of the | | cyclomethicone |
| | | Invention | | (D5) |
| ALLANTOIN | | 0.691953 | | 0.691953 |
| OCTYLDODECANOL | 6.563691 | 6.563691 | 6.563691 | 6.563691 |
| HYDROGENATED JOJOBA OIL | 5.63 | 5.63 | 5.63 | 5.63 |
| ISOSTEARYL ISOSTEARATE | 9.23 | 9.23 | 9.23 | 9.23 |
| TRIDECYL TRIMELLITATE | 18.62 | 18.62 | 0.78 | 0.78 |
| OZOKERITE | | 7.21 | | 7.21 |
| BUTYROSPERMUM PARKII (SHEA | 0.23 | 0.23 | 0.23 | 0.23 |
| BUTTER) | | | | |
| DISTEARDIMONIUM HECTORITE | | 1.036759 | | 1.036759 |
| TITANIUM DIOXIDE (and) | | 1.15 | | 1.15 |
| ALUMINUM HYDROXIDE (and) | | | | |
| STEARIC ACID | | | | |
| HYDROGENATED | 15.4 | 15.4 | 3.26 | 3.26 |
| POLYISOBUTENE | | | | |
| POLYETHYLENE | | 1.27 | | 1.27 |
| NYLON-12 | | 0.92 | | 0.92 |
| HDI/TRIMETHYLOL | | 1.73 | | 1.73 |
| HEXYLLACTONE | | | | |
| CROSSPOLYMER | | | | |
| POLYCYCLOPENTADIENE | | 5.07 | | 5.07 |
| CYCLOPENTASILOXANE | | | 29.98 | 29.98 |
| PHENYL TRIMETHICONE | 5.54 | 5.54 | 5.54 | 5.54 |
| VINYL | | 1.38 | | 1.38 |
| DIMETHICONE/METHICONE | | | | |
| SILSESQUIOXANE | | | | |
| CROSSPOLYMER | | | | |
| DIMETHICONE (and) | | 12.92 | | 12.92 |
| DIMETHICONE CROSSPOLYMER | | | | |
| DIMETHICONE/VINYL | | 2.31 | | 2.31 |
| DIMETHICONE CROSSPOLYMER | | | | |
| PROPYLENE CARBONATE | | 0.057597 | | 0.057597 |
| C12-15 ALKYL BENZOATE | 1.89 | 1.89 | 1.89 | 1.89 |
| OCTYLDODECANOL (and) | | 1.15 | | 1.15 |
| TOCOPHERYL ACETATE (and) | | | | |
| SILICA | | | | |

The comparative example containing cyclopentasiloxane (D5) contained more silicone oil (35.52%) than non-silicone oil (27.58%). The invention example contained more non-silicone oil (57.56%) than silicone oil (5.54%). The invention example yielded a satin finish when applied to skin. In contrast, the comparative example, containing more silicone oil than non-silicone oil, yielded a more matte finish when applied to skin.

### III. PRODUCT EVALUATION ON LIPS

Example E corresponded to Example C except that 13.3% by weight pigments were added.

Example F corresponded to a mousse stick of the following formula:

| INCI Name | % present |
|---|---|
| ALLANTOIN | 0.599868 |
| BHT | 0.05 |
| YELLOW 6 LAKE | 1.13 |
| RED 7 | 1.11 |
| OCTYLDODECANOL | 5.690196 |
| HYDROGENATED JOJOBA OIL | 4.44 |
| ISOSTEARYL ISOSTEARATE | 8 |
| TRIDECYL TRIMELLITATE | 9.22 |
| OZOKERITE | 5.68 |
| BUTYROSPERMUM PARKII (SHEA BUTTER) | 0.20 |
| DISTEARDIMONIUM HECTORITE | 0.900004 |
| FRAGRANCE | 0.20 |
| TITANIUM DIOXIDE (and) ALUMINUM HYDROXIDE | 1 |
| (and) STEARIC ACID | |
| MICA | 9.50 |
| CALCIUM SODIUM BOROSILICATE (and) TITANIUM | 1.26 |
| DIOXIDE (and) IRON OXIDES | |
| HYDROGENATED POLYISOBUTENE | 13.35 |
| POLYETHYLENE | 1 |
| NYLON-12 | 0.80 |
| HDI/TRIMETHYLOL HEXYLLACTONE | 1.50 |
| CROSSPOLYMER | |
| POLYCYCLOPENTADIENE | 4.40 |
| PHENYL TRIMETHICONE | 5.26 |
| VINYL DIMETHICONE/METHICONE SILSESQUIOXANE | 1.20 |
| CROSSPOLYMER | |
| DIMETHICONE (and) DIMETHICONE | 11.20 |
| CROSSPOLYMER | |
| TRIMETHYL PENTAPHENYL TRISILOXANE | 6.92 |
| DIMETHICONE/VINYL DIMETHICONE | 2 |
| CROSSPOLYMER | |
| PROPYLENE CARBONATE | 0.049932 |
| C12-15 ALKYL BENZOATE | 1.64 |
| COCOS NUCIFERA (COCONUT) OIL (and) MINERAL | 0.70 |
| OIL (and) ALOE BARBADENSIS LEAF JUICE | |
| OCTYLDODECANOL (and) TOCOPHERYL | 1 |
| ACETATE (and) SILICA | |

Comparative Example G was the commercial product Maybelline Moisture Extreme.

Wear testing was performed using a panel of six subjects. A color image, using a Chromasphere with Qualiper 1 software, was acquired prior to application and immediately after application. A shine measurement, using an instrument called the SAMBA, was also done immediately after application. The subjects waited one hour, after which chromasphere and shine measurements were acquired. The subjects then ate a meal: the subjects blotted one time with a paper napkin, after which they had one hot drink (coffee or tea), one cold drink (glass of water), 10 mouthfulls of a cheese or ham sandwich, and a whole apple cut in half. After the meal, the final chromasphere (color) measurement was made.

Wear was evaluated using an atlas to score the images for the percentage of color remaining. Migration was scored on a 0-4 scale from the one hour chromasphere images.

Shine was evaluated by averaging the gray level of the shiniest (top 0.5%) of pixels in the image acquired using the SAMBA. This is a unit-less measurement. Above 150 is considered to be shiny, 110-140 is considered to be satin, and under 110 is considered to be matte.

### Results:

### Mean (±Standard Deviation)

| | | % Wear after | | | |
|---|---|---|---|---|---|
| | Sample | Classic | Migration | Immediate | Shine |
| Compositions | Size | Meal Test | 1 Hr | Shine | 1 Hr |
| Example E | 6 | 60.8 (±10.2) | 0.0 (±0.0) | 101 (±15.1) | 102 (±12.2) |
| Mousse Stick | | | | | |
| Example F (674158) | 6 | 57.8 (±12.9) | 0.0 (±0.0) | 119 (±15.4)* | 121 (±15.2)* |
| Mousse Stick | | | | | |
| Comparative | 6 | 28.3 (±7.5) | 0.5 (±0.0) | 152 (±5.1) | 148 (±8.6)* |
| Example G - | | | | | |
| Maybelline Moisture | | | | | |
| Extreme | | | | | |

*Panelist excluded from analysis due to abnormal data

| | % Wear after | | | |
|---|---|---|---|---|
| | Classic | Migration | Immediate | Shine |
| Formulas | Meal Test | 1 Hr | Shine | 1 Hr |
| Example F | 58 | 0.00 | 119 | 121 |
| Comparative Example G | 61 | 0.00 | 101 | 102 |

Example E exhibited approximately sixty-one percent (60.8%) wear after the meal test, received a mean score of 0.0 for migration properties, 101 for immediate shine and 102 for shine after 1 hour.

Example F exhibited 58% wear after the meal test, received a mean score of 0.0 for migration properties, 119 for immediate shine and 121 for shine after 1 hour.

What is claimed is:

1. A solid, substantially anhydrous composition capable of imparting a satin-to-matte appearance to keratin materials comprising at least one oil, at least one silicone elastomer, at least one wax, at least one filler, and at least one coloring agent, wherein the composition comprises more non-silicone oil than silicone oil.

2. The composition according to claim 1, wherein the combined total amount of oil present is at least about 30% by weight with respect to the total weight of the composition and the combined total amount of silicone elastomer(s), wax(es), filler(s) and coloring agent(s) present is at least about 30% by weight with respect to the total weight of the composition.

3. The composition according to claim 1, further comprising at least one film forming agent.

4. The composition according to claim 1, wherein the ratio of the total amount of oil present to the combined total amount of silicone elastomer(s), wax(es), filler(s) and coloring agent(s) present ranges from about 2.5: 1 to about 1: 2.5.

5. The composition according to claim 1, wherein the total amount of oil present is at least about 40% by weight with respect to the total weight of the composition and the combined total amount of silicone elastomer(s), wax(es), filler(s) and coloring agent(s) present is at least about 35% by weight with respect to the total weight of the composition.

6. The composition according to claim 1, wherein the composition is substantially free of kaolin, talc and bentonite.

7. The composition according to claim 5, wherein the composition is substantially free of kaolin, talc and bentonite.

8. The composition according to claim 1, comprising 5-30% wax.

9. The composition according to claim 7, comprising 5-30% wax.

10. The composition according to claim 9, comprising 0.5-10% silicone elastomer.

11. A method of producing a solid, substantially anhydrous composition capable of imparting a satin-to-matte appearance to keratin materials comprising combining at least one oil, at least one silicone elastomer, at least one wax, at least one filler, and at least one coloring agent, wherein the composition comprises more non-silicone oil than silicone oil, to form a solid, anhydrous composition.

12. The method according to claim 11, wherein the ratio of the total amount of oil present in the composition to the combined total amount of silicone elastomer(s), wax(es), filler(s) and coloring agent(s) present in the composition ranges from about 2.5:1 to about 1:2.5.

13. The method according to claim 11, wherein the total amount of oil present in the composition is at least about 40% by weight with respect to the total weight of the composition and the combined total amount of silicone elastomer(s), wax(es), filler(s) and coloring agent(s) present in the composition is at least about 35% by weight with respect to the total weight of the composition.

14. The method according to claim 11, wherein the composition is substantially free of kaolin, talc and bentonite.

15. The method according to claim 11, wherein the composition comprises 5-30% wax.

16. The method according to claim 11, wherein the composition comprises 0.5-10% silicone elastomer.

17. A method of delivering a satin-to-matte appearance to a keratin material comprising applying a solid, substantially anhydrous composition comprising at least one oil, at least one silicone elastomer, at least one wax, at least one filler, and at least one coloring agent, wherein the composition comprises more non-silicone oil than silicone oil, to the keratin material in an amount sufficient to deliver a satin-to-matte appearance to a keratin material.

18. The method according to claim 17, wherein the ratio of the total amount of oil present in the composition to the combined total amount of silicone elastomer(s), wax(es), filler(s) and coloring agent(s) present in the composition ranges from about 2.5:1 to about 1:2.5.

19. The method according to claim 17, wherein the total amount of oil present in the composition is at least about 40% by weight with respect to the total weight of the composition and the combined total amount of silicone elastomer(s), wax(es), filler(s) and coloring agent(s) present in the composition is at least about 35% by weight with respect to the total weight of the composition.

20. The method according to claim 17, wherein the composition is substantially free of kaolin, talc and bentonite.

21. The method according to claim 17, wherein the composition comprises 5-30% wax.

22. The method according to claim 17, wherein the keratin material is skin.

23. The method according to claim 17, wherein the keratin material is lips.

24. A method of minimizing drag or cakey feeling associated with application of compositions which impart a satin-to-matte appearance to keratin materials comprising combining at least one oil, at least one silicone elastomer, at least one wax, at least one filler, and at least one coloring agent, wherein the composition comprises more non-silicone oil than silicone oil, to form a solid, substantially anhydrous composition capable of imparting a satin-to-matte appearance to keratin materials.

25. A method of minimizing oil migration associated with a solid composition comprising at least about 30% oil upon application to a keratin material comprising combining at least 30% oil, at least one silicone elastomer, at least one wax, at least one filler, and at least one coloring agent, wherein the composition comprises more non-silicone oil than silicone oil, to form a solid, substantially anhydrous composition.
